(19) United States
(12) Patent Application Publication
(10) Pub. No.: US 2024/0287468 A1
(43) Pub. Date: Aug. 29, 2024

(54) BACTERIOPHAGES AGAINST VANCOMYCIN-RESISTANT ENTEROCOCCI

(71) Applicant: Ferring B.V., Hoofddorp (NL)

(72) Inventors: Kristin WANNERBERGER, Pully (CH); Alexander SULAKVELIDZE, Brookeville, MD (US)

(73) Assignee: Ferring B.V., Hoofddorp (NL)

(21) Appl. No.: 18/570,436

(22) PCT Filed: Jun. 14, 2022
(86) PCT No.: PCT/IB2022/055508

§ 371 Date: Dec. 14, 2023

Related U.S. Application Data
(60) Provisional application No. 63/210,555, filed on Jun. 15, 2021.

(51) Int. Cl. C12N 7/00; A61K 35/741; A61K 35/76; A61K 36/064; A61K 39/00; A61K 39/09; A61P 31/04

(52) U.S. Cl. CPC C12N 7/00; A61K 35/741; A61K 35/76; A61K 36/064; A61K 39/09; A61P 31/04; A61K 2039/521; C12N 2795/00021; C12N 2795/00032

(57) ABSTRACT

Described herein are bacteriophages that infect and lyse Vancomycin-Resistant Enterococci (VRE). The bacteriophages are useful, for example, for the prophylactic or therapeutic treatment of subjects infected with Enterococci, including VRE, or who are at risk of infection by Enterococci, including VRE, and for other uses described herein.

### CROSS-REFERENCE TO RELATED APPLICATIONS

This application claims priority to U.S. Provisional Application No. 63/210,555 filed Jun. 15, 2021, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on Jun. 11, 2021, is named 052209-0383_SL.txt and is 1,952,761 bytes in size.

### FIELD OF THE INVENTION

Described herein are bacteriophages that infect and lyse Vancomycin-Resistant Enterococci (VRE). The bacteriophages are useful, for example, for the prophylactic or therapeutic treatment of subjects infected with Enterococci, including VRE, or who are at risk of infection by Enterococci, including VRE, and for other uses described herein.

### BACKGROUND

### Bacteriophages

Bacteriophages are viruses that infect bacteria and lyse them as part of their replication/lytic cycle. Bacteriophages (or “phages” for short) derive their name from the Greek word “phago” meaning “to eat” or “bacteria eaters,” and were discovered by Felix D′Herelle in the first part of the twentieth century. Phages are specific for their targeted bacterial hosts, and do not infect human or other eukaryotic cells. Bacteriophages have been used therapeutically in humans since 1919 to target pathogenic bacteria. Early enthusiasm led to their use as both prophylaxis and therapy for diseases caused by bacteria. In the U.S. during the 1940s, Eli Lilly commercially manufactured six phage products for human use including preparations targeted against Staphylococci, Streptococci and other respiratory pathogens. With the advent of antibiotics, the therapeutic use of phages gradually fell out of favor in the U.S. and Western Europe and little research was conducted. However, bacteriophage therapy continued to be utilized in Eastern Europe.

### Vancomycin-Resistant Enterococci (VRE)

Enterococci are gram-positive, facultative anaerobic cocci found in soil, food, water, animals, birds, and insects. In humans, they are a common colonizing bacterial species in the human intestinal tract and the genitourinary tracts. Enterococci cause a variety of infections, including urinary tract infections, intra-abdominal and pelvic wound infections, endocarditis, and bacteremia. Vancomycin-resistant Enterococci (VRE) include strains from several different enterococcal species, with clinically significant VRE infections known to be caused by Enterococcus faecium and Enterococcus faecalis. Although the taxonomy of Enterococci has not been finalized, it is generally accepted that the genus consists of 19 species.

Antibiotic management of serious enterococcal infections has always been difficult due to the intrinsic resistance of the organisms to most antimicrobial agents. In the 1970s enterococcal infections were treated with combinations of a cell wall active agent such as penicillin and an aminoglycoside. However, during the 1980s enterococcal strains appeared with high levels of aminoglycoside resistance and resistance to penicillin, mediated both by a plasmid-encoded β-lactamase and by changes in penicillin binding proteins. Such organisms are called VRE because of their resistance to vancomycin; they are also resistant to the penicillin-aminoglyroside combination. Despite the release of two drugs to which VRE are susceptible (quinupristin/dalfopristin and linezolid (see, e.g., Plouffe, Clin. Infect Dis. 31 (Supp. 4): S144-49 (2000)), these microorganisms remain an important cause of morbidity and mortality in immunocompromised patients. Infections caused by VRE have emerged as a particularly serious problem among the elderly, immunocompromised, immunosuppressed and/or seriously ill patients in cancer centers and organ transplant units. VRE are easily transmitted within the hospital environment, and many hospital outbreaks have been reported. Although humans serve as the primary reservoir, the organisms can be readily isolated from the immediate environment of infected/colonized patients (e.g., bedding, furniture, and personal and household items).

Although attention has been focused on the impact of VRE infections on severely ill and immunocompromised patients, intestinal colonization with VRE is becoming relatively common among general hospital patients as well. Once colonized with VRE, a patient may remain colonized for life, and colonized persons who subsequently become immunocompromised, such as due to cancer chemotherapy or immunosuppressive therapies (such as may be adjunct to transplantation), are at risk for developing serious blood or wound infections with VRE.

### Treatments for VRE Infections

VRE bacteria are intrinsically resistant to many antimicrobial agents and developing new antibiotics effective against VRE has proven to be very difficult. The treatment of VRE infections with two antibiotics (quinupristin/dalfopristin and linezolid) approved by the FDA has only been moderately successful, and VRE strains resistant to quinupristin/dalfopristin and linezolid have already been identified. Thus, there remains an urgent need for effective agents against VRE, such as for reducing the risk of or preventing VRE infection and/or for treating patients already infected with VRE.

### SUMMARY OF THE INVENTION

The present invention provides bacteriophages that infect and lyse VRE. The bacteriophages can be formulated in compositions, such as over-the-counter (OTC) or pharmaceutical compositions, for use for reducing the risk of, preventing, or treating VRE infections, can be used to generate vaccine/bacterin compositions, and can be used in assays to detect VRE.

Thus, there are provided isolated bacteriophage that infect and lyse one or more strains of vancomycin-resistant enterococci (VRE), wherein the bacteriophage is of a strain selected from: (i) bacteriophage strain VREML237-2 deposited with the ATCC under Accession Number PTA-126934, or a variant strain thereof, wherein the variant strain has an average nucleotide identity across its genome (gANI) of at least 80% to said bacteriophage strain VREML237-2; (ii) bacteriophage strain VREML110-1 deposited with the ATCC under Accession Number PTA-126932, or a variant strain thereof, wherein the variant strain has a gANI of at least 80% to said bacteriophage strain VREML110-1; (iii) bacteriophage strain VREML105 deposited with the ATCC under Accession Number PTA-126931, or a variant strain thereof, wherein the variant strain has a gANI of at least 80% to said bacteriophage strain VREML105; (iv) bacteriophage strain VREML202-1 deposited with the ATCC under Accession Number PTA-126933, or a variant strain thereof, wherein the variant strain has a gANI of at least 80% to said bacteriophage strain VREML202-1; (v) bacteriophage strain VREML85-2 deposited with the ATCC under Accession Number PTA-126930, or a variant strain thereof, wherein the variant strain has a gANI of at least 80% to said bacteriophage strain VREML85-2; (vi) bacteriophage strain VREML110-2 deposited with the ATCC under Accession Number PTA-127012 or a variant strain thereof, wherein the variant strain has a gANI of at least 80% to said bacteriophage strain VREML110-2; (vii) bacteriophage strain VREML237-1 deposited with the ATCC under Accession Number PTA-127016 or a variant strain thereof, wherein the variant strain has a gANI of at least 80% to said bacteriophage strain VREML237-1; (viii) bacteriophage strain VREML85-1 deposited with the ATCC under Accession Number PTA-127011 or a variant strain thereof, wherein the variant strain has a gANI of at least 80% to said bacteriophage strain VREML85-1; (ix) bacteriophage strain VREML 137-2 deposited with the ATCC under Accession Number PTA-127013 or a variant strain thereof, wherein the variant strain has a gANI of at least 80% to said bacteriophage strain VREML137-2; (x) bacteriophage strain VREML202-2 deposited with the ATCC under Accession Number PTA-127015 or a variant strain thereof, wherein the variant strain has a gANI of at least 80% to said bacteriophage strain VREML202-2; and (xi) bacteriophage strain VREML 139 deposited with the ATCC under Accession Number PTA-127014 or a variant strain thereof, wherein the variant strain has a gANI of at least 80% to said bacteriophage strain VREML 139.

The isolated bacteriophage may be a variant strain of one of said deposited bacteriophage strains that has a gANI of at least 90% to the deposited bacteriophage strain. The isolated bacteriophage may be a variant strain of one of said deposited bacteriophage strains that has a gANI of at least 95% to the deposited bacteriophage strain. The isolated bacteriophage may be a variant strain of one of said deposited bacteriophage strains that has an RFLP DNA profile substantially equivalent to the RLFP DNA profile of the deposited bacteriophage strain.

Also provided are isolated progeny bacteriophages of a deposited bacteriophage strain as disclosed herein, wherein the isolated progeny bacteriophage has a gANI of ≥80% relative to the deposited bacteriophage strain. The isolated progeny bacteriophage may have a gANI of ≥95% relative to the deposited bacteriophage strain. The isolated progeny bacteriophage may habe a gANI of ≥98% or ≥99.9% relative to the deposited bacteriophage strain.

Also provided are compositions comprising bacteriophages of one or more of the bacteriophage strains disclosed herein and a pharmaceutically acceptable carrier. The one or more bacteriophage strains in the composition may comprise or consist of (i) bacteriophage strain VREML237-2 deposited with the ATCC under Accession Number PTA-126934, or a variant strain thereof, wherein the variant strain has a gANI of at least 80% to said bacteriophage strain VREML237-2; (ii) bacteriophage strain VREML110-1 deposited with the ATCC under Accession Number PTA-126932, or a variant strain thereof, wherein the variant strain has a gANI of at least 80% to said bacteriophage strain VREML110-1; (iii) bacteriophage strain VREML105 deposited with the ATCC under Accession Number PTA-126931, or a variant strain thereof, wherein the variant strain has a gANI of at least 80% to said bacteriophage strain VREML105; (iv) bacteriophage strain VREML202-1 deposited with the ATCC under Accession Number PTA-126933, or a variant strain thereof, wherein the variant strain has a gANI of at least 80% to said bacteriophage strain VREML202-1; and (v) bacteriophage strain VREML85-2 deposited with the ATCC under Accession Number PTA-126930, or a variant strain thereof, wherein the variant has a gANI of at least 80% to said bacteriophage strain VREML85-2. The one or more bacteriophage strains in the composition may comprise or consist of (i) bacteriophage strain VREML237-2; (ii) bacteriophage strain VREML110-1; (iii) bacteriophage strain VREML105; (iv) bacteriophage strain VREML202-1; and (v) bacteriophage strain VREML85-2.

The composition may be provided in an oral dosage form, optionally provided with an enteric coating, optionally in a form selected from tablets, hard gel capsules, soft gel capsules, dragees, powders, granules, solutions, suspensions, dispersions, syrups, and microgels. The composition may be provided in a rectal dosage form, optionally in a suppository, enema, rectal foam, lotion, or gel. The composition may be provided in a vaginal dosage form, optionally in a suppository, cream vaginal foam, lotion, or gel. The composition may be provided in a topical dosage form, optionally in an emulsion, cream, lotion, gel, or spray. The composition may be provided in a pulmonary dosage form, optionally in a powder, aerosol, nebulizer, or insufflator composition. The composition may be provided in an injectable dosage form.

In any embodiments, the composition optionally further comprises a probiotic. The probiotic may comprise a probiotic bacteria, such as one or more selected from the group consisting of L. acidophilus, L. rhamnosus, L. gasseri, L. reuteri, L. bulgaricus, L. plantarum, L. johnsonii, L. paracasei, L. casei, L. salivarius, L. lactis, B. bifidum, B. longum, B. breve, B. infantis, B. lactis, B. adolescentis, Streptococcus thermophilus, Bacillus cerus, Bacillus subtilis, and any combinations thereof. The probiotic may comprise a probiotic yeast, such one or more of Saccharomyces cerevisiae, Saccharomyces boulardii, Saccharomyces cerevisiae var. boulardii, Issatchenkia occidentalis, Lachancea thermotolerans, Metschnikowia ziziphicola, Torulaspora delbrueckii, and any combination thereof.

Also provided are methods of reducing the risk of, or preventing, or treating VRE colonization or infection in a subject in need thereof, or for modulating a human subject's microbiome, comprising administering to the subject a bacteriophage or composition as disclosed herein.

Also provided are the bacteriophages and compositions as disclosed herein for use in reducing the risk of, or preventing, or treating VRE colonization or infection in a subject in need thereof, or for modulating a human subject's microbiome.

Also provided are uses of a bacteriophage or composition as disclosed herein in the preparation of a medicament for reducing the risk of, or preventing, or treating VRE colonization or infection in a subject in need thereof, or for modulating a human subject's microbiome.

In accordance with any such method, composition for use, or use, the subject may be at risk of colonization or infection by VRE or be colonized or infected by VRE, and optionally may be an immunosuppressed or immunocompromised subject. In accordance with any such method, composition for use, or use, the subject may suffer from VRE gut colonization and the method may be effective to reduce or eliminate VRE gut colonization.

In some embodiments of a method, bacteriophage or composition for use, or use as disclosed herein, the treatment further comprises administering a probiotic to the subject, optionally wherein the probiotic is provided in the same composition as the bacteriophage.

In some embodiments of a method, bacteriophage or composition for use, or use as disclosed herein, the bacteriophage or composition or medicament is administered orally.

In some embodiments of a method, bacteriophage or composition for use, or use as disclosed herein, the bacteriophage, composition, or medicament is administered rectally.

In some embodiments of a method, bacteriophage or composition for use, or use as disclosed herein, the bacteriophage, composition, or medicament is administered topically.

In some embodiments of a method, bacteriophage or composition for use, or use as disclosed herein, bacteriophage, composition, or medicament is administered vaginally.

Also provided are compositions comprising a lytic enzyme produced by a bacteriophage as disclosed herein.

Also provided are compositions comprising a derivative product of bacteriophage as disclosed herein, wherein the derivative product has activity against VRE or encodes a product that has activity against VRE, optionally wherein the derivative product is one or more selected from DNA, cDNA, mRNA and synthetic polynucleotide sequences, DNA/RNA hybrids, and anti-sigma factor genes and expression products thereof.

Also provided are vaccines comprising a VRE bacterial lysate obtained by lysing a VRE strain with a bacteriophage as disclosed herein or a lytic enzyme thereof; methods for vaccinating a subject against VRE infection comprising administering to the subject such a vaccine; such VRE bacterial lysates for use in vaccinating a subject against VRE infection, and uses of such VRE bacterial lysates in the preparation of a medicament for vaccinating a subject against VRE infection.

Also provided are methods for detecting VRE in a sample, comprising treating the sample with a bacteriophage as disclosed herein or a lytic enzyme thereof, thereby specifically inducing release of a measurable VRE bacterial product, and measuring the released VRE bacterial product, optionally wherein the released VRE bacterial product is one or more of adenosine triphosphate (ATP) and protein kinase (AKT). In some embodiments, the sample is a fecal sample obtained from a subject.

### BRIEF DESCRIPTION OF THE DRAWINGS

FIG. 1. shows reference DNA RFLP profiles of the bacteriophages described herein.

FIG. 2. shows results of an assessment of the efficacy of a bacteriophage cocktail as described herein in an in vitro human gut simulator model.

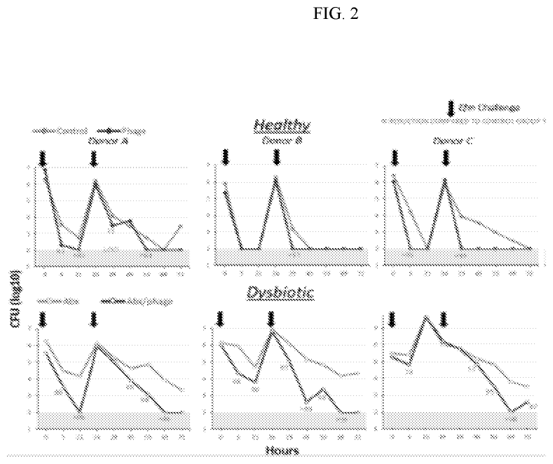

### DETAILED DESCRIPTION OF THE INVENTION

As noted above, described herein are bacteriophages that target (i.e., infect and lyse, e.g., produce lytic infection in) VRE. The bacteriophages can be formulated in compositions, such as over-the-counter (OTC) or pharmaceutical compositions, for use for reducing the risk of, preventing, or treating VRE infections, can be used to generate vaccine/bactrin compositions, and can be used in assays to detect VRE. The bacteriophages and compositions comprising them are useful for preventing or reducing infection by VRE, including preventing or reducing gut colonization by VRE, thereby preventing or reducing the risk of infection by VRE or treating VRE. Without being bound by theory, it is understood that therapy using the bacteriophages and compositions described herein are safe and effective through specific and targeted prevention or reduction of VRE infection, and thereby reduce invasive disease due to VRE and reduce the incidence, prevalence and transmission of VRE infection, while maintaining a healthy gut microbiome by specifically targeting VRE and avoiding side effects associated with broad-spectrum antibiotic therapies.

### Definitions

Technical and scientific terms used herein have the meanings commonly understood by one of ordinary skill in the art to which the present invention pertains, unless otherwise defined. Any suitable materials and/or methods known to those of ordinary skill in the art can be utilized in carrying out the present invention in view of the guidance provided herein; however, specific materials and methods are described for illustrative purposes. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

As used herein, “about” when used with a numerical value means the numerical value stated as well as plus or minus 10% of the numerical value. For example, “about 10” should be understood as both “10” and “9-11.”

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the term “comprising” means that the described compositions, methods, or kits include at least the stated elements, and may include other elements that are not specified.

As used herein, the phrases “effective amount” and “therapeutically effective amount” used with reference to a therapeutically active agent (e.g., a phage) means an amount that provides the specific pharmacological effect for which the agent is administered to a subject in need of such treatment, e.g., to reduce the risk of, prevent, or reduce VRE infection (or VRE colonization). It is emphasized that a therapeutically effective amount will not always be effective to prevent or reduce VRE infection (or VRE colonization) in a given subject, even though such amount is deemed to be a therapeutically effective amount by those of skill in the art. The therapeutically effective amount may vary based on the specific active agent, route of administration and dosage form, age and weight of the subject, and/or the subject's condition, including the type and severity of the VRE infection.

The terms “individual,” “subject,” and “patient” are used interchangeably herein, and refer to any individual mammalian subject, including human subjects, including male and female human subjects.

“Preventing” as used herein means to reduce the risk of VRE infection or VRE colonization or prevent VRE infection or VRE colonization, or to reduce the level of VRE infection or VRE colonization, in a subject in need thereof.

“Treating” as used herein means to reduce the level of VRE infection in a subject in need thereof, including to reduce the VRE infection to undetectable levels or to eliminate VRE colonization.

The term VRE “colonization” is used herein to refer to the presence of VRE in a subject that is not necessarily causing disease in the subject.

The term VRE “infection” is used herein to refer to VRE invasion of a subject's tissue that is causing disease in the subject.

A “Vancomycin-resistant enterococci (VRE) strain” as used herein refers to a VRE strain (isolate) with a minimum inhibitory concentration to vancomycin of at least 16 μg/ml, which may be assessed by an assay as described in the examples below. See, e.g., National Committee for Clinical Laboratory Procedures, “Methods for Dilution Antimicrobial Susceptibility Tests for Bacteria that Grow Aerobically.” (3rd ed., 1993) (Nat. Committee for Clin. Lab. Standards, Villanova Pa.); National Committee for Clinical Laboratory Standards, “Performance Standards for Antimicrobial Disk Susceptibility Tests.” (5th ed., 1993) (Nat. Committee for Clin. Lab. Standards, Villanova Pa.). VREs may belong to the species Enterococcus faecium, Enterococcus faecalis, E. gallinarum, E. casseliflavus, E. durans, E. avium, and E. raffinosis. VRE targeted by the phages described herein typically may present in one or more of the gastrointestinal tract, skin, and genital tract, but can also be found in other parts of the body. Non-limiting examples of diseases and conditions that may be caused by VRE include bacteremia (e.g., sepsis), meningitis, pneumonia, endocarditis, urinary tract infections, intra-abdominal infections (e.g., peritonitis), pelvic infections, skin and soft tissue infections (including wound infections), and nosocomial infections.

As used herein, a “variant” strain of a bacteriophage strain described herein has at least 80% average nucleotide identity across its genome (“gANI”) to a reference bacteriophage strain. gANI is a similarity index between a given pair of genomes. Typically, scholars would identify organisms with a gANI≥95% as belonging to the “Same Species.” See Olm M., “Are these microbes the ‘same’?” microBEnet (2; 2017) (available at microbe.net/2017/02/15/are-these-microbes-the-same/), and Jain et al. (Jain et al. Nature Comm. 9(1):5114 (2018). A variant strain having at least 90% gANI to the reference strain is presumed and deemed to have the same phenotypic characteristics as said reference bacteriophage strain. A “variant” strain may be obtained independently of the reference strain, may be a progeny of the reference strain, or may be a recombinant derivative of the reference strain (e.g., made recombinantly using the reference strain or one or more or all of its genomic sequences as starting material).

As used herein, “isolated” as used with reference to a bacteriophage means the bacteriophage has been removed from its original environment in which it naturally occurs. An “isolated” bacteriophage may have been purified from, cultivated separately from, or cultured separately from the environment in which it is naturally located.

### Bacteriophages

Provided herein are isolated bacteriophages of each of the following (eleven) bacteriophage strains and variant strains thereof having at least 80%, at least 90%, or at least 95% gANI to the deposited strains. The genomic sequence of each strain is set forth in the Sequence Listing incorporated herein by reference:
VREML237-2 (SEQ ID NO: 1) deposited at the ATCC under Accession Number PTA-126934
VREML110-1 (SEQ ID NO: 2) deposited at the ATCC under Accession Number PTA-126932
VREML 105 (SEQ ID NO: 3) deposited at the ATCC under Accession Number PTA-126931
VREML202-1 (SEQ ID NO: 4) deposited at the ATCC under Accession Number PTA-126933
VREML85-2 (SEQ ID NO: 5) deposited at the ATCC under Accession Number PTA-126930
VREML110-2 (SEQ ID NO: 6) deposited at the ATCC under Accession Number PTA-127012
VREML237-1 (SEQ ID NO: 7) deposited at the ATCC under Accession Number PTA-127016
VREML85-1 (SEQ ID NO: 8) deposited at the ATCC under Accession Number PTA-127011
VREML 137-2 (SEQ ID NO: 9) deposited at the ATCC under Accession Number PTA-127013
VREML202-2 (SEQ ID NO: 10) deposited at the ATCC under Accession Number PTA-127015
VREML 139 (SEQ ID NO: 11) deposited at the ATCC under Accession Number PTA-127014

These isolated deposited strains and variants thereof having at least 80%, at least 90%, or at least 95% gANI to the deposited strains are referred to collectively in the discussion herein as bacteriophages (or phages) “as described herein.”

As illustrated in the examples, each of these strains specifically targets (infects and lyses) one or more strains of VRE, and are useful for reducing the risk of infection, preventing infection, and/or treating infection by one or more strains of VRE, including for reducing or eliminating colonization by one or more strains of VRE, and for other uses described herein. For example, as illustrated in the examples, each of these strains specifically targets and lyses one or more of 214 VRE strains isolated from human subjects, and one or more of five reference Enterococci isolates: E401 (ATCC 51299), E403 (ATCC 19433), E618 (ATCC 700221), E400 (ATCC 29212) and E402 (ATCC 11823).

As noted above, as used herein “variants” (or “variant strains”) include progeny obtained by cultivating bacteriophages of the deposited strains, as illustrated in the examples below. Restriction Fragment Length Polymorphism (RFLP) can be used to identify bacteriophage strains or their progeny. The progeny strain may have a substantially equivalent DNA RFLP profile to the DNA RFLP profile of the original bacteriophage strain as defined in Tenover et al., J. Clin. Microbiol. 33(9): 2233-39 (1995). As reported in Tenover, when genomes of identically propagated organisms are electrophoretically analyzed following restriction enzyme digestion, some variation in RFLP profiles may be seen. Tenover describes a system for interpreting chromosomal DNA RFLP profiles using Pulsed-Field Gel Electrophoresis (PFGE). In particular, Tenover sets forth various categories of genetic and epidemiologic relatedness including for identifying organisms that are “indistinguishable” from or “closely related” to each other. As used herein, a bacteriophage strain is “substantially equivalent” to a reference bacteriophage strain if the RLFP profiles are “indistinguishable” or “closely related” under the criteria of Tenover, supra.

Thus, a “progeny” strain may have a DNA RFLP profile substantially equivalent to the DNA RFLP profile of the parent deposited bacteriophage strain, as illustrated in the examples. A progeny may have ≥80% gANI to the parent deposited bacteriophage. A progeny may have ≥90% gANI to the parent deposited bacteriophage. A progeny may have ≥95% gANI to the parent deposited bacteriophage. A progeny may have ≥98% gANI to the parent deposited bacteriophage. A progeny may have ≥99% gANI to the parent deposited bacteriophage.

As noted above, variant strains having at least 90% gANI to a reference bacteriophage strain are presumed and deemed to have the same phenotypic characteristics as the reference bacteriophage strain. Thus, the present disclosure includes variants of these deposited strains having at least 90% gANI to a reference deposited strain and other variants having the same phenotypic characteristics as a deposited strain.

Also provided herein are derivative products of an isolated bacteriophage as described herein. A “derivative product” as used herein refers to substances that constitute subunits or expression products of the bacteriophages described herein, including (but not limited to) nucleic acids, partial or complete genes, lytic enzymes and other gene expression products, and other structural components of a bacteriophage, such as polyribonucleotide(s) and polydeoxyribonucleotide(s), including modified or unmodified bacteriophage DNA, cDNA, mRNA and synthetic polynucleotide sequences, as well as DNA/RNA hybrids, such as derivative products that have activity against VRE or encode products that have activity against VRE. Thus, one or more derivative products of the bacteriophages described herein may be included in the compositions described herein and used in the methods described herein.

Anti-sigma factor genes and expression products thereof are examples of such derivative products. One or more of the phages described herein may encode an anti-sigma factor gene that inhibits bacterial transcriptional activity of the host (target) VRE. See, e g., Hughes, et al. Ann. Rev Microbiol 52:231-86 (1998). The anti-sigma factor gene grants such phages an enhanced ability to inhibit transcription within target (VRE) bacterial cell, thereby enhancing the phage's ability to lyse the target bacterial cell. Thus, anti-sigma factor genes and expression products thereof are examples of derivative products that may be included in the compositions described herein and used in the prophylactic and therapeutic treatment methods described herein, such as to enhance the potency of the composition and/or the efficacy of the method.

Lytic enzymes are another example of phage derivative products described herein. The bacteriophages described herein encode one or more lytic enzymes involved in lysing the host (target) VRE. Phage lytic enzymes are produced by bacteriophages either as part of their virion to facilitate bacterial infection through local peptidoglycan degradation, or as soluble proteins to induce massive cell lysis at the end of the lytic replication cycle. See, e.g., Briers, Viruses 11(2): 113 (2019). Lytic enzymes can cause rapid lysis of their targeted bacteria, resulting in significant reduction or elimination of the targeted bacterium's levels. In addition to enhancing the potency or efficacy of the compositions and prophylactic and therapeutic treatment methods described herein, lytic enzymes can be used in detection assays for rapid identification of specific bacteria. See, e.g., Nelson, et al., “Using bacteriophage lytic enzymes as a diagnostic tool for rapid identification of specific bacteria.” In American Society for Microbiology General Meeting, Salt Lake City, Utah (2002). Lytic enzymes for use in the compositions and methods described herein may be isolated from cultures of the phages described herein, or prepared by recombinant techniques. Thus, lytic enzymes may be included in the compositions described herein and used in the prophylactic and therapeutic treatment methods and detection methods described herein, such as to enhance the potency of the composition and/or the efficacy of the treatment method, or to detect specific target VRE bacteria.

### Compositions

Also provided herein are compositions comprising one or more of the isolated bacteriophage strains described herein. Compositions comprising two or more isolated bacteriophage strains as described herein are referred to herein as a bacteriophage “cocktail.” As noted above, the compositions may be OTC compositions or pharmaceutical compositions. For example, the composition may be a pharmaceutical composition, dietary supplement, nutraceutical composition, nutritional supplement, or probiotic composition.

A bacteriophage cocktail may comprise any combination of two or more of the isolated bacteriophage strains described herein, including any two or more of the following (eleven) deposited strains (or variants thereof having at least 80%, at least 90%, or at least 95% gANI to the deposited strains, including variants having the same phenotypic characteristics, as determined by phenotypic assay or by having at least 90% gANI to a deposited bacteriophage strain):
VREML237-2 (SEQ ID NO: 1) deposited at the ATCC under Accession Number PTA-126934
VREML110-1 (SEQ ID NO: 2) deposited at the ATCC under Accession Number PTA-126932
VREML 105 (SEQ ID NO: 3) deposited at the ATCC under Accession Number PTA-126931
VREML202-1 (SEQ ID NO: 4) deposited at the ATCC under Accession Number PTA-126933
VREML85-2 (SEQ ID NO: 5) deposited at the ATCC under Accession Number PTA-126930
VREML110-2 (SEQ ID NO: 6) deposited at the ATCC under Accession Number PTA-127012
VREML237-1 (SEQ ID NO: 7) deposited at the ATCC under Accession Number PTA-127016
VREML85-1 (SEQ ID NO: 8) deposited at the ATCC under Accession Number PTA-127011
VREML137-2 (SEQ ID NO: 9) deposited at the ATCC under Accession Number PTA-127013
VREML202-2 (SEQ ID NO: 10) deposited at the ATCC under Accession Number PTA-127015
VREML 139 (SEQ ID NO: 11) deposited at the ATCC under Accession Number PTA-127014.

As one specific example, a bacteriophage cocktail may comprise strains of two or more or all of the following (five) isolated bacteriophage strains (or variants thereof having at least 80%, at least 90%, or at least 95% gANI to the deposited strains, including variants having the same phenotypic characteristics, as determined by phenotypic assay or by having at least 90% gANI to a deposited bacteriophage strain):
VREML237-2 (SEQ ID NO: 1) (deposited at the ATCC under Accession Number PTA-126934)
VREML110-1 (SEQ ID NO: 2) (deposited at the ATCC under Accession Number PTA-126932)
VREML 105 (SEQ ID NO: 3) (deposited at the ATCC under Accession Number PTA-126931)
VREML202-1 (SEQ ID NO: 4) (deposited at the ATCC under Accession Number PTA-126933) and
VREML85-2 (SEQ ID NO: 5) (deposited at the ATCC under Accession Number PTA-126930)

As a further specific example, a bacteriophage cocktail may comprise strains of each of the following (five) isolated bacteriophage strains (or variants thereof having at least 80%, at least 90%, or at least 95% gANI to the deposited strains, including variants having the same phenotypic characteristics, as determined by phenotypic assay or by having at least 90% gANI to a deposited bacteriophage strain):
VREML237-2 (SEQ ID NO: 1) (deposited at the ATCC under Accession Number PTA-126934)
VREML110-1 (SEQ ID NO: 2) (deposited at the ATCC under Accession Number PTA-126932)
VREML105 (SEQ ID NO: 3) (deposited at the ATCC under Accession Number PTA-126931)
VREML202-1 (SEQ ID NO: 4) (deposited at the ATCC under Accession Number PTA-126933) and
VREML85-2 (SEQ ID NO: 5) (deposited at the ATCC under Accession Number PTA-126930).

As a further specific example, a bacteriophage cocktail may comprise strains of each of the following (five) isolated bacteriophage strains:
VREML237-2 (SEQ ID NO: 1) (deposited at the ATCC under Accession Number PTA-126934)
VREML110-1 (SEQ ID NO: 2) (deposited at the ATCC under Accession Number PTA-126932)
VREML 105 (SEQ ID NO: 3) (deposited at the ATCC under Accession Number PTA-126931)
VREML202-1 (SEQ ID NO: 4) (deposited at the ATCC under Accession Number PTA-126933) and
VREML85-2 (SEQ ID NO: 5) (deposited at the ATCC under Accession Number PTA-126930).

The specific combination of isolated bacteriophage strains described herein used in a given bacteriophage cocktail can be selected based on the target strain(s) of VRE. For example, an isolated bacteriophage strain or combination of two or more isolated bacteriophage strains described herein can be tested in vitro against the target strain(s) of VRE to confirm efficacy prior to using strain or the combination in a cocktail to treat a specific subject or group of subjects. Suitable screening methodologies are known in the art and illustrated in the examples herein. For use in such testing, target strain(s) of VRE can be obtained from clinical specimens obtained from a specific subject or group of subjects, from the environment of a specific subject or group of subjects, or identified and obtained from other sources. Suitable methodologies are known in the art and illustrated in the examples herein.

Also provided are compositions that comprise isolated bacteriophage strains bacteriophages of one or more bacteriophage strains as described herein and further comprise one or more probiotics. As used herein, a “probiotic” includes probiotic bacteria and probiotic yeast. Suitable probiotic bacteria are known in the art and include Lactobacillus species (such as L. acidophilus, L. rhamnosus, L. gasseri, L. reuteri, L. bulgaricus, L. plantarum, L. johnsonii, L. paracasei, L. casei, L. salivarius, and L. lactis), Bifidobacterium species (such as B. bifidum, B. longum, B. breve, B. infantis, B. lactis, and B. adolescentis), and Streptococcus thermophilus, Bacillus cerus, and Bacillus subtilis. Suitable probiotic yeast are known in the art and include Saccharomyces cerevisiae, Saccharomyces boulardii, Saccharomyces cerevisiae var. boulardii, Issatchenkia occidentalis, Lachancea thermotolerans, Metschnikowia ziziphicola, and Torulaspora delbrueckii.

Thus, in some embodiments, a composition as described herein comprises one or more isolated bacteriophage strains bacteriophage strains as described herein and one or more probiotics. In specific embodiments, a composition as described herein comprises one or more isolated bacteriophage strains bacteriophage strains as described herein and one or more strains of probiotic bacteria. In other specific embodiments, a composition as described herein comprises one or more isolated bacteriophage strains bacteriophage strains as described herein and one or more strains of probiotic yeast. In yet other specific embodiments, a composition as described herein comprises one or more isolated bacteriophage strains bacteriophage strains as described herein and one or more strains of probiotic bacteria and one or more strains of probiotic yeast. Without wanting to be bound by theory, it is believed that the positive health impact of the VRE bacteriophages described herein may be further enhanced when combined with a probiotic.

As one specific example, a composition as described herein may comprise two or more or all of the following isolated bacteriophage strains bacteriophage strains (or variants thereof as described above) and one or more probiotics:
VREML237-2 (SEQ ID NO: 1) (deposited at the ATCC under Accession Number PTA-126934)
VREML110-1 (SEQ ID NO: 2) (deposited at the ATCC under Accession Number PTA-126932)
VREML105 (SEQ ID NO: 3) (deposited at the ATCC under Accession Number PTA-126931)
VREML202-1 (SEQ ID NO: 4) (deposited at the ATCC under Accession Number PTA-126933) and
VREML85-2 (SEQ ID NO: 5) (deposited at the ATCC under Accession Number PTA-126930).

The one or more probiotics may be one or more probiotic bacteria selected from Lactobacillus species (such as L. acidophilus, L. rhamnosus, L. gasseri, L. reuteri, L. bulgaricus, L. plantarum, L. johnsonii, L. paracasei, L. casei, L. salivarius, and L. lactis), Bifidobacterium species (such as B. bifidum, B. longum, B. breve, B. infantis, B. lactis, and B. adolescentis), and Streptococcus thermophilus, Bacillus cerus, and Bacillus subtilis; one or more probiotic yeast selected from Saccharomyces cerevisiae, Saccharomyces boulardii, Saccharomyces cerevisiae var. boulardii, Issatchenkia occidentalis, Lachancea thermotolerans, Metschnikowia ziziphicola, and Torulaspora delbrueckii, or any combination thereof.

As noted above, in any embodiments, a composition comprising an isolated bacteriophage and probiotic as described herein may be an OTC composition or a pharmaceutical composition.

Additionally or alternatively, a composition as described herein may comprise one or more phage derivative products as described herein. In some embodiments, a composition as described herein includes one or more lytic enzymes produced by one or more of the bacteriophages formulated in the composition. In some embodiments, a composition as described herein additionally or alternatively comprises one or more anti-sigma factor genes or expression products thereof of one or more of the bacteriophages formulated in the composition. As noted above, in any embodiments, a composition comprising a bacteriophage and a phage derivative product (such as lytic enzyme(s)) as described herein may be an OTC composition or a pharmaceutical composition.

An OTC or pharmaceutical composition as described herein comprises the isolated bacteriophage(s) (and, in some embodiments, probiotic(s) and/or phage derivative products such as lytic enzyme(s)) and one or more pharmaceutically acceptable carriers, and, optionally one or more pharmaceutically acceptable excipients. Suitable carriers and excipients for bacteriophage(s) are known in the art. Typically, a pharmaceutically acceptable carrier is a pharmaceutically-acceptable, non-toxic carrier, filler, or diluent, suitable for use as a vehicle for formulating pharmaceutical compositions for administration to the target patient (animal or human) by the intended route of administration.

Pharmaceutically acceptable excipients, also referred to as auxiliary agents or accessory ingredients, encompass those conventionally used in pharmaceutical compositions for administration to the target patient (animal or human) by the intended route of administration, such as, but not limited to, matrix-forming agents, thickeners, binders, lubricants, pH adjusting agents, protecting agents, viscosity enhancers, wicking agents, disintegrants, including non-effervescent and effervescent disintegrants, surfactants, antioxidants, wetting agents, colorants, flavoring agents, taste-masking agents, sweeteners, preservatives and so forth. In addition to being pharmaceutically acceptable, the auxiliary agents typically will be selected to be compatible with the other ingredients of the composition, including the bacteriophage(s) and probiotic(s) (if present).

The OTC and pharmaceutical compositions described herein may be formulated for any suitable route of administration (which may depend on the site of VRE colonization or infection). Phage compositions for different routes of administration have been disclosed. See, e.g., Qadir et al., Brazilian J. Pharm. Sci. (2018) 54(1). For example, phages can be formulated for routes of administration including oral, buccal, sublingual, rectal, nasal, topical, otic, vaginal, bronchial, pulmonary, or parenteral (including subcutaneous, intramuscular, intravenous, intradermal, intraperitoneal, intrapleural, intravesicular and intrathecal) administration, or for administration via an implant, or urinary tract rinse or catheter. The composition or route of administration may be selected to provide a targeted effect of one or more bacteriophages described herein, and may depend on the site(s) of colonization or infection.

The OTC and pharmaceutical compositions may be prepared by any suitable method for making the dosage form at issue, with such methods being well known in the art of pharmacy. Such methods typically will include the step of bringing in association one or more bacteriophages as described herein (and, optionally, one or more probiotics and/or phage derivative products such as lytic enzyme(s)) and a pharmaceutically acceptable carrier, and, optionally, one or more pharmaceutically acceptable auxiliary agents.

In some embodiments, a composition as described herein is formulated for oral administration and administered orally. Dosage forms for OTC and pharmaceutical compositions suitable for oral administration are known in the art, and include discrete dosage forms such as tablets (including chewable tablets), hard gel capsules containing a dry phage-containing composition, soft liquid gel capsules containing a liquid phage-containing composition, and dragees, and bulk dosage forms such as powders, granules, solutions, suspensions, dispersions, syrups, microgels, and the like.

Dosage forms of OTC and pharmaceutical compositions formulated for oral administration may be formulated to protect the phages from the acidic environment of the stomach. For example, compositions formulated for oral administration may be provided with or comprise a coating, such as an enteric coating or delayed release coating (such as in the form of coated tablets or capsules, or granules provided with a coating) to protect the bacteriophage (and, optionally the probiotic(s) and/or phage derivative products such as lytic enzyme(s)) and maintain viability during passage through the acidic environment of stomach, or may be microencapsulated (such as in alginate-chitosan microspheres). Suitable coating and microencapsulation materials are known in the art. Additionally or alternatively, compositions formulated for oral administration be may be formulated with or administered with an agent that reduces stomach acids (e.g., a gastric acid reducing agent), such as gastric acid neutralizing agent (e.g., bicarbonate, such as sodium bicarbonate), or a proton pump inhibitor (e.g., omeprazole).

Additionally, or alternatively, such compositions may be provided with or comprise a delayed release coating to provide release of viable phages (and, optionally the probiotic(s) and/or phage derivative products such as lytic enzyme(s)) at a desired site of release, such as in the small intestine.

For example, a composition as described herein formulated for oral administration may comprise one or more of the following ingredients: water, such as deionized water, pharmaceutical grade water, or mineral water; sodium chloride, sodium bicarbonate; a buffer solution (such as Tris-HCl at pH 7.0-7.5); sweeteners, such as sucrose (e.g., a 5% sucrose solution), trehalose, maltodextrin, glycerol, dextran, and sorbitol, cellulose; thickeners, such as tapioca, dextrin, gellan gum, and gelatin; and other excipients such as hydroxypropyl methylcellulose, poly(acrylic acid) (“PAA”), poly(ethyleneglycol) (“PEG”), and casein, and combinations of any two or more thereof.

As one specific, non-limiting example, a composition as described herein may be formulated in a 0.9% sodium chloride solution, optionally to provide about 1×10¹⁰ PFU phage in 1 mL of a 0.9% sodium chloride solution. Such a composition optionally may be mixed with bicarbonate water, such as 15-50 mL bicarbonate water, just prior to administration, for consumption.

In alternative embodiments, a composition as described herein is formulated for rectal administration. Dosage forms for OTC and pharmaceutical compositions suitable for rectal administration are known in the art, and may include a suppository, rectal foam, lotion, gel or enema for rectal administration.

In alternative embodiments, a composition as described herein is formulated for vaginal administration. Dosage forms for OTC and pharmaceutical compositions suitable for vaginal administration are known in the art, and may include a suppository, cream, vaginal foam, lotion, gel, vaginal rinse, or catheter for vaginal administration.

In alternative embodiments, a composition as described herein is formulated for parenteral administration, such as by infusion or injection, including subcutaneous, intramuscular, intravenous, intradermal, intraperitoneal, intrapleural, intravesicular, and intrathecal injection. Compositions suitable for parenteral administration are known in the art, and may include sterile aqueous and non-aqueous compositions formulated for infusion or injection. Such compositions may be provided in unit-dose or multi-dose containers, for example pre-filled syringes, sealed vials, sealed ampoules, or sealed pouches. Such compositions may be ready-to-use, or may be freeze-dried (lyophilized) or spray-dried compositions that are reconstituted prior to use with a sterile liquid carrier, for example, water.

In alternative embodiments, a composition as described herein is formulated for topical administration. Dosage forms for OTC and pharmaceutical compositions suitable for topical administration are known in the art, and include solutions, emulsions, creams, lotions, gels, and sprays. For example, the composition as described herein may be formulated as a gel for topical administration.

In alternative embodiments, a composition as described herein is formulated for pulmonary or bronchial administration, such as by oral or nasal inhalation. Dosage forms for compositions suitable for pulmonary administration are known in the art, and include powder compositions and fine particle formulations (e.g., dusts or mists) which may be generated by and administered via metered dose pressurized aerosols, nebulizers, insufflators, or the like.

### Treatments

The bacteriophages and compositions described herein can be used in prophylactic and therapeutic treatment methods to reduce the risk of or prevent VRE colonization or infection or to treat VRE colonization or infection, or to modulate a subject's microbiome (such as by preventing or reducing colonization by VRE), or for reducing or eliminating VRE gut colonization, or for treating VRE-related diseases or conditions, or for similar effects.

In accordance with such uses, the bacteriophages and compositions described herein can be administered according to any effective dosing regimen, which may vary based on the prophylactic or therapeutic effect to be achieved (e.g., prevention of infection vs. treatment of colonization or treatment of bacteremia), the specific bacteriophage(s) used, the route of administration and dosage form, and patient characteristics such as one or more of the age and weight of the patient, the patient's condition, and the type and severity of the VRE infection. Exemplary dosages are described below for illustrative purposes only.

A typical bacteriophage dose for a human patient is likely to contain from 10³ to 10¹² plaque forming units (PFU) of the one or more bacteriophage(s), including from 10⁴ to 10¹¹, such as 10⁶ to 10¹⁰, which may be administered one or more times per day, such as one, two, three, four, or five times per day. The dose may be provided in a single discrete dosage form (e.g., one tablet or capsule) or multiple discrete dosage forms (e.g., two, three, four, or more tablets or capsules), or in a suitable volume of a bulk dosage form (e.g., 1 mL of a liquid composition). The treatment may continue for one or more days, including one day, one week, two weeks, three weeks, one month, or longer.

One example of a suitable dose regimen is 1-3 capsules or tablets, each containing 10⁸-10¹⁰ PFUs bacteriophage(s), orally administered 1-3 times a day for 1-4 weeks. Another example of a suitable dose regimen is 1 mL of a liquid composition containing 10⁸-10¹¹ PFUs bacteriophage(s), orally administered 1-3 times a day for 1-4 weeks.

As noted above, the bacteriophage compositions and methods described herein may be used in conjunction with an agent that reduces stomach acids. For example, a subject undergoing treatment may also be treated with a proton pump inhibitor (e.g., omeprazole), and/or may also consume a gastric acid neutralizer, such as bicarbonate (e.g., sodium bicarbonate) shortly before taking a dose of the bacteriophage compositions.

The bacteriophages and compositions described herein may be used prophylactically or therapeutically to treat any subject in need thereof, including any subject at risk of VRE colonization or infection (including hospitalized subjects) or any subject colonized or infected with VRE.

The bacteriophages and compositions described herein advantageously may be used in subjects facing, being treated with, or recovering from one or more of an invasive medical procedure, chemotherapy, a solid organ transplant, or an immunosuppressive therapy, or other subjects who are immunosuppressed or immunocompromised. In such embodiments, the bacteriophages and compositions described herein may be administered one or more of prior to, during, and after the invasive medical procedure, chemotherapy, solid organ transplant, or immunosuppressive therapy. For example, a course of bacteriophage treatment as described herein may be commenced 1-4 weeks prior to the other treatment period (e.g., the invasive medical procedure, chemotherapy, solid organ transplant, or immunosuppressive therapy) and be continued during and, optionally after, the other treatment period.

### VRE Detection Methods

The bacteriophages described herein (or lytic enzymes thereof) also can be used in in vitro assays to detect target bacteria (e.g., VRE) in biological samples obtained from a subject, such as to diagnose whether a subject is infected with VRE and/or to assess the level of infection (colonization), including to assess the need for or efficacy of treatment as described herein. Without being bound by theory, it is believed the bacteriophages described herein (or their lytic enzymes) specifically lyse the targeted bacteria (e.g., VRE) without affecting other prokaryotic or eukaryotic cells that may be present, thus specifically inducing release of measurable products of the target bacteria, such as adenosine triphosphate (ATP) and/or protein kinase (AKT) specific to the target VRE. Thus, a detection assay can be conducted based on the detection of ATP or AKT of the target bacteria.

An illustrative example of such an assay is as follows. Samples of clinical material (e.g., fecal specimens) to be analyzed are obtained and suspended in an appropriate buffer. One or more bacteriophages (or lytic enzymes thereof) are added to the suspension, as a result of which any targeted bacterial cells present in the samples are lysed and their ATP is released. To detect the ATP (for example), a luciferin+luciferase preparation is added, and luminescence is measured, such as by using a luminometer. A quantitative assay may be developed by creating a calibration curve between the luminometer readings and the number of targeted bacteria cells lysed (in general, the average amount of ATP per bacterial cell is 0.5-1.0 fg). Absence of luminescence indicates absence of targeted bacteria cells in the sample analyzed.

### Vaccines and Bacterins

Also provided are vaccines and bacterins prepared using the bacteriophages (or lytic enzymes thereof) described herein. For example, the bacteriophages described herein (including variants of the deposited strains as discussed above) or lytic enzymes thereof can be used to lyse specific strains of targeted bacteria (e.g., targeted VRE), to obtain bacterial lysates containing immunological epitopes of the bacteria, which in turn can be used to prepare vaccines/bacterins against the targeted VRE. The final vaccine/bacterin preparations can be prepared by methods known in the art. When used to obtain the bacterins, the phages may be removed to obtain the final vaccine/bacterin preparation. Alternatively, the phages may be retained in the final preparation, in a viable, active state. In such embodiments, the phages as present in the vaccine/bacterin formulation may remain active against the targeted bacteria (e.g., targeted VRE), providing another mechanism of action of efficacy of the preparation, e.g., they may lyse target bacteria (VRE) present in the vaccinated subject. For example, the final vaccine/bacterin preparation may be prepared to have phage(s) present at levels ranging from 10³-10¹² PFU/ml. For example, the final vaccine/bacterin preparation may be prepared to have phage(s) present at levels ranging from 10⁶-10¹⁰ PFU/ml.

In specific embodiments, a vaccine/bacterin preparation is prepared against prevalent, problematic strains of the targeted bacteria to obtain vaccine/bacterin preparations containing immunological epitopes that are most relevant for protecting a target patient population against infection. In further specific embodiments, the bacteriophage is retained unaltered in the final vaccine/bacterin preparation as discussed above.

Bacteriophage-based vaccines and bacterins also may be prepared by using recombinant constructs expressing relevant genes of the bacteriophage (e.g., genes associated with lytic activity, such as those encoding lytic enzymes) or isolated or recombinantly produced lytic enzymes, which can be used in place of phages per se in a protocol as outlined above. An example of this general methodology is outlined in Panthel et al., Infect Immun.71(1): 109-16 (2003).

In accordance with any of these embodiments, also provided are uses of vaccines comprising bacterins as described above to immunize a subject in need thereof, such as a subject suffering from or at risk of infection by VRE. Suitable vaccination protocols can be developed by those skilled in the art based on the guidance provided herein.

### Kits

Also provided are kits for practicing the various embodiments described herein.

A kit for prophylactic or therapeutic uses of the bacteriophage(s) described herein may comprise a composition as described herein together with instructions for the prophylactic or therapeutic use of the composition as herein described, optionally together with packaging material.

A kit for prophylactic or therapeutic uses of the vaccine(s)/bacterin(s) described herein may comprise a vaccine/bacterin as described herein together with instructions for the prophylactic or therapeutic use of the vaccine/bacterin as herein described, optionally together with packaging material.

A kit for a VRE detection assay may comprise one or more bacteriophages as described herein (or one or more lytic enzymes thereof) together with instructions for the use of the bacteriophage(s) (or enzyme(s)) in a VRE detection assay as herein described, optionally together with packaging material.

### EXAMPLES

The invention is further described in the following examples, which are not in any way intended to limit the scope of the invention.

### Example 1: VRE Isolates

### Obtaining VRE Isolates

VRE were isolated from patients in the surgical intensive care and intermediate care units of the University of Maryland VA Medical Center in Baltimore. Maryland. Trypticase Soy Agar supplemented with 5% sheep blood (BBL, Cockeysville Md.) was used to isolate enterococci from urine, wounds and sterile body fluids. VRE were isolated from stool specimens on Colistin Nalidixic Acid (CNA) agar (Difco labs, Detroit, Mich.) supplemented with defibrinated sheep blood (5%), vancomycin (10 μg/ml) and amphotericin (1 μg/ml). See Facklam, et al., Enterococcus. In Manual of Clinical Microbiology (6th ed. 1995) (Am. Soc. Microbiol., Washington, D.C.), pp. 308-312.

### Identification of VRE

Enterococci were identified by esculin hydrolysis and growth in 6.5% NaCl at 45° C. Identification to the species level was performed in accordance with Facklam and Collins, J. Clin. Microbiol., 27:731-34 (1989).

### Antimicrobial Susceptibility Testing of VRE

Antimicrobial susceptibilities to ampicillin, vancomycin, streptomycin, and gentamicin were determined using the E test quantitative minimum inhibitory concentration procedure (AB Biodisk, Solna Sweden). Quality control strains of E. faecium (ATCC 29212, ATCC 51299) were used to ensure potency of each antimicrobial agent tested. With the exception of vancomycin, susceptibility interpretations from the National Committee for Clinical Laboratory Standards were adhered to. See National Committee for Clinical Laboratory Procedures, “Methods for Dilution Antimicrobial Susceptibility Tests for Bacteria that Grow Aerobically.” (3rd ed., 1993) (Nat. Committee for Clin. Lab. Standards, Villanova Pa.); National Committee for Clinical Laboratory Standards, “Performance Standards for Antimicrobial Disk Susceptibility Tests.” (5th ed., 1993) (Nat. Committee for Clin. Lab. Standards, Villanova Pa.). A VRE isolate was defined as one having a minimum inhibitory concentration to vancomycin of at least 16 μg/ml.

### Defining Generically Distinct VRE Strains

Distinct VRE isolates were characterized as such by contour-clamped homogeneous electric field electrophoresis after digestion of chromosomal DNA with Smal. See, Verma, P. et al., “Epidemiologic characterization of vancomycin resistant enterococci recovered from a University Hospital” In Abstracts of the 94th General Meeting of the American Society for Microbiology, Las Vegas Nev. (1994); Dean, et al., “Vancomycin resistant enterococci (VRE) of the vanB genotype demonstrating glycoprotein (G) resistance inducible by vancomycin (V) or teicoplanin (T)” In Abstracts of the 94th General Meeting of the American Society for Microbiology, Las Vegas Nev. (1994). For VRE strains which differed only by 1-3 bands after initial analysis, electrophoretic studies were also performed using Apal digestion. See Donabedian, J. Clin. Microbiol., 30; 2757-61 (1992). The vancomycin-resistant genotype (vanA, vanB or vanC) was defined by polymerase chain reaction analysis using specific primers selected from published gene sequences. See Goering, R. V. and the Molecular Epidemiological Study Group “Guidelines for evaluating pulsed field restriction fragment patterns in the epidemiological analysis of nosocomial infections.” Abstract of the Third International Meeting of Bacterial Epidemiological Markers; Cambridge England (1994).

Using these methodologies, 214 distinct VRE strains were identified from the strains isolated at the VA Medical Center.

### Example 2: Bacteriophages

### Isolation of VRE Phages

500 ml water from the Chesapeake Bay (a potential source of phages) was mixed with 100 ml of 10 times concentrated LB broth (Difco Laboratories) (comprising 10 g/L tryptone, 5 g/L yeast extract, 10 g/L NaCl). This water-broth mixture was inoculated with 1 ml of an 18-24-hour LB broth culture of a VRE strain and incubated at 37° C. for 24 hours to enrich the mixture any bacteriophage present which can infect the added VRE strain. After incubation, the mixture was centrifuged at 5,000 g for 15 minutes to eliminate matter which may interfere with subsequent filtration. The supernatant was filtered through a 0.45 μm Millipore filter. The filtrate was assayed by the Streak Plate Method and Appelman Tube Turbidity Test to assess lytic activity against different VRE strains, generally using the methodology outlined below.

### Plaque Assay:

A plaque assay can be used to assess whether a given VRE strain is susceptible to infection by a given bacteriophage preparation. Plaque picking can be used to obtain a preparation of a single strain of bacteriophage.

An 18-24-hour nutrient broth culture of the VRE strain (0.1 ml) to be tested and dilutions of a VRE bacteriophage preparation (1.0 ml) are mixed and then added to 4.5 ml 0.7% molten agar in nutrient broth at 45° C. This mixture is completely poured into a petri dish containing 25 ml of nutrient broth solidified with 2% agar, and incubated overnight at 37° C. During overnight incubation at 37° C., VRE grow in the agar and form a confluent lawn with some VRE cells being infected with bacteriophage. The phages replicate and lyse the initially infected cells and subsequently infect and lyse neighboring bacteria. The agar limits the physical spread of the phages throughout the plate, resulting in small, visibly clear areas on the plate called plaques, where bacteriophage has destroyed VRE within the confluent lawn of VRE growth.

The number of plaques formed from a given volume of a given dilution of bacteriophage preparation is a reflection of the titer of the bacteriophage preparation. One plaque with a distinct morphology represents one phage particle that replicated in VRE in that area of the bacterial lawn. Thus, a pure bacteriophage preparation can be obtained by removing the material in that plaque with a pipette, such as a Pasteur pipette (referred to as a “plaque pick”) and using this material as the inoculum for further growth cycles of that phage. By conducting further plaque assays on preparations of phage grown from the same plaque pick, all plaques will have a single appearance (plaque morphology) which is the same as the plaque picked, which is a further indication of purity. Therefore, plaque assay techniques can be used to assess bacteriophage potency against the VRE, and also to assess bacteriophage purity of the bacteriophage preparation.

### Streak Plate Method:

The streak plate method can be used to screen one or more phages against one or more VRE strains.

Eighteen-hour LB broth cultures of one or more enterococci strains to be tested are grown at 37° C. (resulting in approximately 10⁹ CPU/ml) and a loopful of each culture is streaked across a nutrient agar plate in a single line. This results in each plate having a number of different VRE strains streaked across it in single straight lines of growth.

Single drops of bacteriophage filtrates to be tested are applied to each VRE streak, and the plate is incubated for 6 hours at 37° C., at which time the streaks of the different VRE strains are examined for the ability of the phage to form clear areas devoid of bacterial growth, indicating lysis of that particular VRE strain by that particular phage. The VRE host range for a given phage filtrate can be ascertained by determining on which VRE streaks it caused a clear area devoid of growth and on which VRE streaks it did not.

### Appelman Tube Turbidity Test

The Appleman Tube Turbidity test (see Adams, Bacteriophages. (Interscience Publ. New York N.Y.) (1959)) can be used to screen a phage preparation against one or more VRE strains.

18-hour LB broth cultures of one or more VRE strains are prepared, and 0.1 ml of phage filtrate or a dilution thereof is added to 4.5 ml of VRE broth cultures and incubated at 37° C. for 4 hours (monophage preparation containing a single phage), or for 4-18 hours (polyvalent preparation containing at least two phages). Phage-free VRE broth cultures are used as controls. Broth cultures which are normally turbid due to bacterial growth are examined for the ability of the phage preparation to lyse the VRE strain as indicated by clearing of culture turbidity. The host range of a given phage preparation can be ascertained by which VRE broth cultures the phage preparation clears and which broth cultures it does not.

The bacteriophages described herein were isolated and identified using these methodologies.

### Example 3: Characterization of Phages

### Restriction Fragment Length Polymorphism (RFLP) Profile

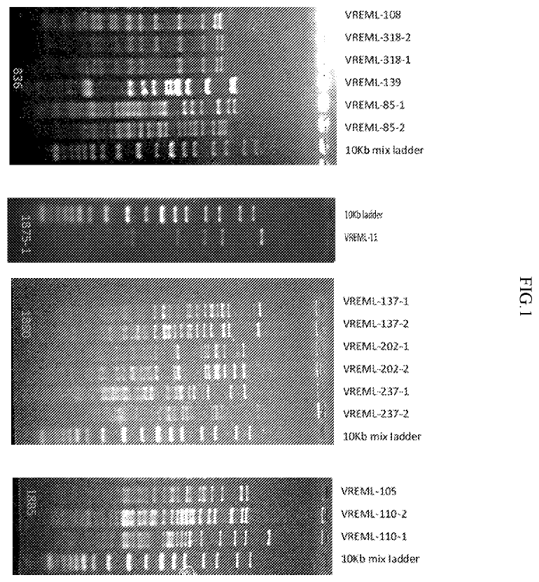

RFLP profiles of the bacteriophages described herein are shown in FIG. 1. DNA was isolated from the bacteriophage using Qiagen Plasmid Miniprep or Midiprep kits (Valencia, CA) according to the manufacturer's directions. The DNA was quantitated by measuring absorbance at 260 nm. Approximately 0.5-1 μg of DNA was digested with an appropriate restriction enzyme (Hind III-digest), and RFLP profile was determined on 1% agarose gel after staining with ethidium bromide.

### Genome Analysis and Average Nucleotide Identity of the VRE Phages

Full genome sequencing and sequence analysis can be used to identify the bacteriophages described herein or their progeny. Progeny that have an average nucleotide identity (ANI)≥95% are considered to be of the “Same Species” as defined by Olm (Olm M., “Are these microbes the ‘same’?” microBEnet (2; 2017) (available at microbe.net/2017/02/15/are-these-microbes-the-same/) and Jain et al. (Jain et al. Nature Comm. 9(1):5114 (2018). A phage having a gANI≥95% to a reference phage is deemed to be “substantially equivalent” to the reference bacteriophage.

The VRE bacteriophages were sequenced on MiSeq with read length 2×250 bp. Reads were trimmed for illumina adapter, length (≥50 bp), quality (q≥20) and mapped to Enterococci reference sequences available in GenBank. The unmapped reads were collected and assembled using Unicycler assembler. The strain delineation was assessed by calculating average nucleotide identity over genome (gANI). See, e.g., Jain et al., supra; Varghese et al., Nucleic Acids Res. 43(14):6761-71 (2015).

### Example 4: Production of Phage Preparations

The VRE bacteriophages described herein can be propagated as illustrated herein.

Strains of the target bacteria (e.g., VRE) or other closely related bacterial species on which the bacteriophage can propagate are cultured in batch culture using a suitable growth medium (e.g., BHI broth), and inoculated with the bacteriophage at a pre-determined multiplicity of infection (MOI). Following incubation and bacterial lysis, the bacteriophage is harvested and purified and/or concentrated to yield phage progeny suitable for the uses described herein.

Suitable purification and concentration procedures include one or more of filtration, centrifugation (including continuous-flow centrifugation), size-exclusion chromatography, ion exchange chromatography, and other known bacteriophage purification and concentration techniques. See, e.g., Adams MH. Bacteriophages. 443-519 (Interscience Publishers, Ltd, London) (1959). The purity of the phage preparation can be assessed by one or more of electron microscopy, SDS-PAGE, DNA restriction digest, and analytical ultracentrifugation.

The bacteriophage concentration of a preparation can be adjusted using phage titration protocols. For example, the bacteriophage concentration of a preparation is determined. If a more concentrated phage preparation is desired, concentration is increased by one or more of filtration, centrifugation, or other means. If a less concentrated phage preparation is desired, the concentration is reduced by dilution with water or other pharmaceutically acceptable diluent (such as a pharmaceutically acceptable buffer). Typical pharmaceutical or OTC compositions include a phage titer of 10⁶ to 10¹² PFU/mL, such as a phage titer of 10⁹ to 10¹¹ PFU/mL.

Bacteriophage preparations may be stored at 2-8° C. Alternatively, phage preparations can be freeze-dried or spray-dried for storage, or can be encapsulated and stabilized by approaches known in the art, such as with one or more of a protein, lipid, and polysaccharide. Upon reconstitution, the phage titer can be verified using phage titration protocols, host bacteria assays, or other widely known bacteriophage assay techniques. See, e.g., Adams, supra.

### Example 5: Phage Activity

Bacteriophages of the bacteriophage strains described herein effectively lyse VRE strains, demonstrating their efficacy against VRE strains.

The VRE phages described herein can be used alone or in various combinations in order to target specific VRE strains or strain subgroups. For example, the following phages of the following five strains can be combined in one cocktail to effectively target all of the 214 VRE strains isolated at the VA Medical Center, as well as five reference Enterococci isolates: E401 (ATCC 51299), E403 (ATCC 19433), E618 (ATCC 700221), E400 (ATCC 29212) and E402 (ATCC 11823):
VREML237-2 (SEQ ID NO: 1) (deposited at the ATCC under Accession Number PTA-126934)
VREML110-1 (SEQ ID NO: 2) (deposited at the ATCC under Accession Number PTA-126932)
VREML 105 (SEQ ID NO: 3) (deposited at the ATCC under Accession Number PTA-126931)
VREML202-1 (SEQ ID NO: 4) (deposited at the ATCC under Accession Number PTA-126933)
VREML85-2 (SEQ ID NO: 5) (deposited at the ATCC under Accession Number PTA-126930).

As set forth in tables below, this five-phage cocktail lyses 94% of the 214 VRE strains isolated at the VA Medical Center plus the five reference strains.

TABLE 1
Activity of five VRE phages against 214 VRE strains

| | | | | | | Kill Rate If Added |
|---|---|---|---|---|---|---|
| | | | Resis- | Un- | | To Cocktail |
| Rank | Phage | Kills | tant | tested | Unique | (cumulative) |
| 1 | VREML- | 186 | 26 | 2 | 186 | 186 (86.92%)1 |
| | 237-2 | | | | | (VREML 237-2 |
| | | | | | | only) |
| 2 | VREML- | 158 | 54 | 2 | 7 | 193 (90.19%) |
| | 85-2 | | | | | (+VREML-85-2) |
| 3 | VREML- | 169 | 43 | 2 | 4 | 197 (92.06%) |
| | 110-1 | | | | | (+VREML-110-1) |
| 4 | VREML- | 68 | 144 | 2 | 2 | 199 (92.99%) |
| | 202-1 | | | | | (+VREML-202-1) |
| 5 | VREML- | 146 | 66 | 2 | 1 | 200 (93.46%) |
| | 105 | | | | | (+VREML-105; |
| | | | | | | all 5 present) |

TABLE 2
Activity of five VRE phages against
five ATCC Enterococci strains

| | | Number of | |
|---|---|---|---|
| Strain, | Strain, | Effective | |
| ITX ID | ATCC ID | Phages | Phage names |
| E401 | ATCC 51299 | 5 | VREML-85-2, VREML-202-1, |
| | | | VREML-237-2, VREML-105, |
| | | | VREML-110-1 |
| E403 | ATCC 19433 | 4 | VREML-85-2, VREML-202-1, |
| | | | VREML-237-2, VREML-110-1 |
| E618 | ATCC 700221 | 4 | VREML-85-2, VREML-237-2, |
| | | | VREML-105, VREML-110-1 |
| E400 | ATCC 29212 | 3 | VREML-85-2, VREML-202-1, |
| | | | VREML-237-2 |
| E402 | ATCC 11823 | 3 | VREML-85-2, VREML-237-2, |
| | | | VREML-110-1 |

The PhageSelector™ software used for this analysis is described in Cieplak, et al., Gut Microbes 9(5): 01-19 (2018).

### Example 6: Efficacy in In Vivo Mouse Model

Efficacy of the described bacteriophages was demonstrated in an in vivo mouse model as described herein.

Establishment of sustained VRE colonization in an animal model was carried out as follows:

ICR outbred mice (6 weeks old) were decolonized for three days with 1 mg/ml of gentamicin in drinking water plus subcutaneous injections of clindamycin (2.4 mg/day/mouse) to reduce their normal intestinal flora. Twenty-four hours prior to administration of VRE, antibiotics were stopped to allow for wash out. VRE (about 2×10³ CFU) diluted in saline was administered once by oral gavage to each mouse. Starting one hour after VRE administration, animals were treated with 0.2 mL of the five-phage cocktail described herein at about 1×10⁹ PFU/dose (n=10) or PBS (n=5) by oral gavage every 8 hours over 7 days for a total of 21 doses. Fresh stool pellets (1/mouse) were collected daily to quantitate fecal bacteria levels. The fecal pellets were placed in sterile pre-weighed Eppendorf tubes, weighed, serially diluted in 0.9% saline and plated on Enterococcosel™ Agar (EA), a selective medium for enterococci, with 8 μg/mL of nitrofurantoin (NIT8) (EA NIT8) and erythromycin 32 μg/mL Colony forming units (CFUs) were counted after incubation at 37° C. for 48 hours and used to calculate the log 10 CFU/g feces.

The efficacy of phage treatment to reduce VRE gastrointestinal colonization was determined by quantitating VRE daily in weighed fecal samples from mice in the phage cocktail-treated group compared to the PBS-treated control group. Administration of the phage cocktail reduced the overall load of VRE gastrointestinal colonization compared to the PBS control group by about 0.5 log after phage administration, with the largest reduction in fecal VRE burden achieved by day 6. VRE colonization trended lower in the phage cocktail-treated group than the PBS controls at all time points tested.

### Example 7: Efficacy in In Vitro Human Gut Simulator Model

Efficacy of the described bacteriophages was demonstrated in an in vitro human gut simulator model as described herein. In particular, a five-phage cocktail of bacteriophages VREML237-2, VREML110-1, VREML105, VREML202-1, and VREML85-2 was shown to be effective against VRE in the SHIME® model (Simulator of the Human Intestinal Microbial Ecosystem or SHIME®), as outlined below. See Moye et al, J. Food Prot. 82(8): 1336-1349 (2019).

Healthy, VRE-negative human donor stool from 3 subjects was used in the SHIME® model, which simulates the ascending, transverse and descending colon using inoculum preparation, retention time, pH, temperature settings and reactor feed composition. The SHIME® setup was adapted to include 12 proximal colon compartments, to test the impact of inter-individual variability of 3 donors. SHIME® systems using healthy donor stool from 3 VRE-negative healthy individuals were stabilized for 2 weeks under healthy or dysbiotic (i.e., treated with 33.9 ppm clindamycin for 7 days (days −8 to −2)) conditions. Ascending colonic reactors were treated with either PBS or the phage cocktail (6×10¹⁰ PFU) twice a day, 8 hours apart, for 5 days starting on day-1. Four hours after the third and fifth dose of PBS or phage cocktail, the reactors were challenged with VRE (1×10⁹ CFU) at 0 hours (day 0) and 24 hours (day 1).

Efficacy of the phage cocktail under both healthy and dysbiosed (i.e., following antibiotic treatment) conditions was compared to PBS treatment. Efficacy was calculated by quantitation of the VRE burden (CFU) over time (1 hour after each PBS or phage cocktail treatment, and at the time of each VRE challenge) in the proximal colon. The phage cocktail was effective in reducing the VRE burden to levels of reduction >50% compared to controls (PBS or clindamycin). In 3 out of 3 healthy stools, the phage cocktail accelerated E. faecium clearance and reduced E. faecium abundance by >83-99% within 21 hours after challenge compared to placebo control. In 3 out of 3 dysbiotic stool (healthy donor stool treated with clindamycin), the phage cocktail accelerated E. faecium clearance and reduced E. faecium abundance by >51-99% within 21 hours after challenge compared to antibiotic-treated samples. Effects on overall endogenous Enterococcus abundance was limited, indicating specific antipathogenic targeting of E. faecium VRE by the phages in the phage cocktail. In all samples, phage treatment did not impact short-chain fatty acid levels, and/or it improved levels of beneficial biomarkers compared to dysbiotic (antibiotic-treated) samples. Additionally, the phage cocktail did not alter the endogenous microbial composition in healthy donors and in 2 out of 3 donors the cocktail stimulated the microbial recovery of primary substrate degraders following antibiotic treatment.

### Example 8: Phase 1/2A Clinical Trial

Phase 1: This double-blinded, placebo-controlled, randomized Phase 1 trial will assess safety in about 10-20 healthy adults of a single dose of a VRE phage cocktail as described herein (i.e., a composition containing bacteriophages of two or more of the VRE bacteriophage strains described herein, such as two or more or all of VREML237-2, VREML110-1, VREML105, VREML202-1, and VREML85-2) administered orally 2 or 3 times per day over a 7-day period, with up to 3 months of follow up compared to a placebo control group. For example, about 1×10¹⁰ PFU phages may be administered orally two or three times a day, at least one hour before a meal. Clinical safety assessments will include observation of clinical signs and symptoms, analysis of blood parameters, and measuring fecal calprotectin content. The primary objective is to determine the safety of oral administration of a course of the bacteriophage cocktail. The exploratory objectives include determining the impact of the phage cocktail on the human fecal microbiome using state-of-the-art next generation sequencing and bioinformatic techniques, and evaluating phage shedding.
Phase 2a: This double-blinded, placebo-controlled, randomized Phase 2a trial will assess the safety and efficacy of a VRE phage cocktail as described herein (i.e., a composition containing bacteriophages of two or more of the VRE bacteriophage strains described herein, such as two or more or all of VREML237-2, VREML110-1, VREML105, VREML202-1, and VREML85-2) administered orally 2 or 3 times per day over a 2-week period, compared to a placebo control group in about 30-50 VRE-colonized adult subjects, with up to 6 months of outpatient follow up. For example, about 1×10¹⁰ PFU phages may be administered orally two or three times a day, at least one hour before a meal. Clinical safety assessments will be as described for Phase 1 above. Efficacy will be determined by quantitative assessment of VRE fecal shedding. The primary objectives are safety and efficacy as assessed by the impact of phage administration on the levels of VRE in the GI tract vs. placebo. The exploratory objectives include evaluating phage shedding and determining the impact of the phage cocktail on the human gut microbiome.

1. An isolated bacteriophage that infects and lyses one or more strains of vancomycin-resistant Enterococci (VRE), wherein the bacteriophage is of a strain selected from:
(i) bacteriophage strain VREML237-2 (SEQ ID NO: 1) deposited with the ATCC under Accession Number PTA-126934, or a variant strain thereof, wherein the variant strain has an average nucleotide identity across its genome (gANI) of at least 80% to said bacteriophage strain VREML237-2;
(ii) bacteriophage strain VREML110-1 (SEQ ID NO: 2) deposited with the ATCC under Accession Number PTA-126932, or a variant strain thereof, wherein the variant strain has a gANI of at least 80% to said bacteriophage strain VREML110-1;
(iii) bacteriophage strain VREML105 (SEQ ID NO: 3) deposited with the ATCC under Accession Number PTA-126931, or a variant strain thereof, wherein the variant strain has a gANI of at least 80% to said bacteriophage strain VREML105;
(iv) bacteriophage strain VREML202-1 (SEQ ID NO: 4) deposited with the ATCC under Accession Number PTA-126933, or a variant strain thereof, wherein the variant strain has a gANI of at least 80% to said bacteriophage strain VREML202-1;
(v) bacteriophage strain VREML85-2 (SEQ ID NO: 5) deposited with the ATCC under Accession Number PTA-126930, or a variant strain thereof, wherein the variant strain has a gANI of at least 80% to said bacteriophage strain VREML85-2;
(vi) bacteriophage strain VREML110-2 (SEQ ID NO: 6) deposited with the ATCC under Accession Number PTA-127012 or a variant strain thereof, wherein the variant strain has a gANI of at least 80% to said bacteriophage strain VREML110-2;
(vii) bacteriophage strain VREML237-1 (SEQ ID NO: 7) deposited with the ATCC under Accession Number PTA-127016 or a variant strain thereof, wherein the variant strain has a gANI of at least 80% to said bacteriophage strain VREML237-1;
(viii) bacteriophage strain VREML85-1 (SEQ ID NO: 8) deposited with the ATCC under Accession Number PTA-127011 or a variant strain thereof, wherein the variant strain has a gANI of at least 80% to said bacteriophage strain VREML85-1;
(ix) bacteriophage strain VREML137-2 (SEQ ID NO: 9) deposited with the ATCC under Accession Number PTA-127013 or a variant strain thereof, wherein the variant strain has a gANI of at least 80% to said bacteriophage strain VREML137-2;
(x) bacteriophage strain VREML202-2 (SEQ ID NO: 10) deposited with the ATCC under Accession Number PTA-127015 or a variant strain thereof, wherein the variant strain has a gANI of at least 80% to said bacteriophage strain VREML202-2; and
(xi) bacteriophage strain VREML139 (SEQ ID NO: 11) deposited with the ATCC under Accession Number PTA-127014 or a variant strain thereof, wherein the variant strain has a gANI of at least 80% to said bacteriophage strain VREML139.

2. An isolated bacteriophage of claim 1, wherein the bacteriophage is a variant strain of one of said deposited bacteriophage strains that has a gANI of at least 90% to the deposited bacteriophage strain.

3. An isolated bacteriophage of claim 1, wherein the bacteriophage is a variant strain of one of said deposited bacteriophage strains that has a gANI of at least 95% to the deposited bacteriophage strain.

4. An isolated bacteriophage of claim 1, wherein the bacteriophage is a variant strain of one of said deposited bacteriophage strains that has an RFLP DNA profile substantially equivalent to the RLFP DNA profile of the deposited bacteriophage strain.

5. An isolated progeny bacteriophage of a deposited bacteriophage strain of claim 1, wherein the isolated progeny bacteriophage has a gANI of ≥80% relative to the deposited bacteriophage strain.

6. An isolated progeny bacteriophage of a deposited bacteriophage strain of claim 1, wherein the isolated progeny bacteriophage has a gANI of ≥95% relative to the deposited bacteriophage strain.

7. An isolated progeny bacteriophage of a deposited bacteriophage strain of claim 1, wherein the isolated progeny bacteriophage has a gANI of ≥98% or ≥99.9% relative to the deposited bacteriophage strain.

8. A composition comprising:
(i) bacteriophages of one or more strains of any one of claims 1 to 7; and
(ii) a pharmaceutically acceptable carrier.

9. A composition according to claim 8, wherein the one or more bacteriophage strains comprise or consist of:
(i) bacteriophage strain VREML237-2 deposited with the ATCC under Accession Number PTA-126934, or a variant strain thereof, wherein the variant strain has a gANI of at least 80% to said bacteriophage strain VREML237-2;
(ii) bacteriophage strain VREML110-1 deposited with the ATCC under Accession Number PTA-126932, or a variant strain thereof, wherein the variant strain has a gANI of at least 80% to said bacteriophage strain VREML110-1;
(iii) bacteriophage strain VREML 105 deposited with the ATCC under Accession Number PTA-126931, or a variant strain thereof, wherein the variant strain has a gANI of at least 80% to said bacteriophage strain VREML105;
(iv) bacteriophage strain VREML202-1 deposited with the ATCC under Accession Number PTA-126933, or a variant strain thereof, wherein the variant strain has a gANI of at least 80% to said bacteriophage strain VREML202-1; and
(v) bacteriophage strain VREML85-2 deposited with the ATCC under Accession Number PTA-126930, or a variant strain thereof, wherein the variant has a gANI of at least 80% to said bacteriophage strain VREML85-2.

10. A composition according to claim 8, wherein the one or more bacteriophage strains comprise or consist of (i) bacteriophage strain VREML237-2; (ii) bacteriophage strain VREML110-1; (iii) bacteriophage strain VREML105; (iv) bacteriophage strain VREML202-1; and (v) bacteriophage strain VREML85-2.

11. A composition according to any one of claims 8-10, wherein the composition is provided in an oral dosage form, optionally provided with an enteric coating, optionally in a form selected from tablets, hard gel capsules, soft gel capsules, dragees, powders, granules, solutions, suspensions, dispersions, syrups, and microgels.

12. A composition according to any one of claims 8-10, wherein the composition is provided in dosage form selected from:
a rectal dosage form, optionally in a suppository, enema, rectal foam, lotion, or gel;
a vaginal dosage form, optionally in a suppository, cream vaginal foam, lotion, or gel;
a pulmonary dosage form, optionally in a powder, aerosol, nebulizer, or insufflator composition;
a topical dosage form, optionally in an emulsion, cream, lotion, gel, or spray, and an injectable dosage form.

13. A composition according to any one of claims 8-12, wherein the composition further comprises a probiotic.

14. A composition according to claim 13, wherein the probiotic comprises a probiotic bacteria.

15. A composition according to claim 14, wherein the probiotic bacteria is one or more selected from the group consisting of L. acidophilus, L. rhamnosus, L. gasseri, L. reuteri, L. bulgaricus, L. plantarum, L. johnsonii, L. paracasei, L. casei, L. salivarius, L. lactis, B. bifidum, B. longum, B. breve, B. infantis, B. lactis, B. adolescentis, Streptococcus thermophilus, Bacillus cerus, Bacillus subtilis, and any combinations thereof.

16. A composition according to any one of claims 13-15, wherein the probiotic comprises a probiotic yeast.

17. A composition according to claim 16, wherein the probiotic yeast is selected from the group consisting of Saccharomyces cerevisiae, Saccharomyces boulardii, Saccharomyces cerevisiae var. boulardii, Issatchenkia occidentalis, Lachancea thermotolerans, Metschnikowia ziziphicola, Torulaspora delbrueckii, and any combination thereof.

18. A method of reducing the risk of, or preventing, or treating VRE colonization or infection in a subject in need thereof, or for modulating a human subject's microbiome, comprising administering to the subject a bacteriophage according to any one of claims 1-7 or a composition according to of any one of claims 8-17.

19. A bacteriophage according to any one of claims 1-7 or a composition according to of any one of claims 8-17, for use in reducing the risk of, or preventing, or treating VRE colonization or infection in a subject in need thereof, or for modulating a human subject's microbiome.

20. Use of a bacteriophage according to any one of claims 1-7 or a composition according to of any one of claims 8-17, in the preparation of a medicament for reducing the risk of, or preventing, or treating VRE colonization or infection in a subject in need thereof, or for modulating a human subject's microbiome.

21. The method of claim 18, bacteriophage or composition for use of claim 19, or use of claim 20, wherein the subject is at risk of colonization or infection by VRE or is colonized or infected by VRE, optionally wherein the subject is an immunosuppressed or immunocompromised subject.

22. The method, bacteriophage or composition for use, or use of any one of claims 18-21, wherein the subject suffers from VRE gut colonization and the method is effective to reduce or eliminate VRE gut colonization.

23. The method, bacteriophage or composition for use, or use of any one of claims 18-22, wherein the treatment further comprises administering a probiotic to the subject, optionally wherein the probiotic is provided in the same composition as the bacteriophage.

24. The method, bacteriophage or composition for use, or use of any one of claims 18-23, wherein the bacteriophage or composition or medicament is administered orally.

25. The method, bacteriophage or composition for use, or use of any one of claims 18-23, wherein the bacteriophage, composition, or medicament is administered rectally.

26. The method, bacteriophage or composition for use, or use of any one of claims 18-23, wherein the bacteriophage, composition, or medicament is administered topically.

27. The method, bacteriophage or composition for use, or use of any one of claims 18-23, wherein the bacteriophage, composition, or medicament is administered vaginally.

28. A composition comprising a lytic enzyme produced by a bacteriophage according to any one of claims 1-7.

29. A composition comprising a derivative product of bacteriophage according to any one of claims 1-7, wherein the derivative product has activity against VRE or encodes a product that has activity against VRE, optionally wherein the derivative product is one or more selected from DNA, cDNA, mRNA and synthetic polynucleotide sequences, DNA/RNA hybrids, and anti-sigma factor genes and expression products thereof.

30. A vaccine comprising a VRE bacterial lysate obtained by lysing a VRE strain with a bacteriophage of any one of claims 1-7 or a lytic enzyme thereof.

31. A method for vaccinating a subject against VRE infection, comprising administering to the subject a vaccine of claim 30.

32. A VRE bacterial lysate obtained by lysing a VRE strain with a bacteriophage of any one of claims 1-7 or a lytic enzyme thereof, for use in vaccinating a subject against VRE infection.

33. Use of a VRE bacterial lysate in the preparation of a medicament for vaccinating a subject against VRE infection, wherein the bacterial lysate is obtained by lysing a VRE strain with a bacteriophage of any one of claims 1-7 or a lytic enzyme thereof.

34. A method for detecting VRE in a sample, comprising treating the sample with a bacteriophage of any one of claims 1-7 or a lytic enzyme thereof, thereby specifically inducing release of a measurable VRE bacterial product, and measuring the released VRE bacterial product.

35. The method of claim 34, wherein the released VRE bacterial product is one or more of adenosine triphosphate (ATP) and protein kinase (AKT).

36. The method of any one of claims 34-35, wherein the sample is a fecal sample obtained from a subject.
